(19) United States
(12) Patent Application Publication
(10) Pub. No.: US 2016/0220715 A1
(43) Pub. Date: Aug. 4, 2016

(54) METHODS FOR SANITIZING AN INDOOR SPACE USING A PHOTO-HYDRO-IONIZATION GENERATOR

(71) Applicant: Pavel Mazac, Clyde Hill, WA (US)

(72) Inventor: Pavel Mazac, Clyde Hill, WA (US)

(21) Appl. No.: 15/009,260

(22) Filed: Jan. 28, 2016

Related U.S. Application Data
(60) Provisional application No. 62/109,833, filed on Jan. 30, 2015.

(51) Int. Cl. A61L 2/14; A61L 9/22

(52) U.S. Cl. CPC A61L 2/14; A61L 9/22

(57) ABSTRACT

The present invention generally relates to methods of using an air purifier to perform a photo-hydro-ionization (PHI) process to generate an oxidizer plasma to be exhausted into an indoor space. The oxidizer plasma may include ionized-hydro-peroxides (IHPs), superoxide, hydroxyl ions or some combination thereof (hereinafter generally referred to as IHPs). In one embodiment, the process sanitizes the ambient environment of the indoor space by purifying air, sanitizing hard surfaces, and decontaminating the environment by creating and maintaining an elevated level of IHPs at or above 0.02 parts per million (ppm) within the entire indoor space.

### PRIORITY CLAIM

The present application claims the benefit of the filing date of U.S. Provisional Patent Application No. 62/109,833, and wherein its subject matter is incorporated by reference herein in its entirety.

### FIELD OF THE INVENTION

The present invention generally relates to methods for sanitizing an indoor space with at least one air purification unit, and more specifically methods elevating and maintaining a level of ionized-hydro-peroxides, superoxides, hydroxyl ions, or some combination thereof within the indoor space.

### SUMMARY

In one aspect of the present invention, a method for saturating a space with an air purification unit includes the steps of (1) drawing ambient air from the space through a filter located within the unit; (2) drawing air over an ionized-hydro-peroxide generator, located in the unit, to impart oxidizer plasma into the drawn air; (3) purifying the drawn air within the unit with the imparted oxidizer plasma; (4) adding free ionized-hydro-peroxide oxidizer plasma to the purified air to generate saturated air; (5) exhausting the saturated air from the unit; (6) elevating a level of ionized-hydro-peroxides of the air within the indoor space; and (7) maintaining the air within the indoor space to have a level of ionized-hydro-peroxides of at least 0.02 parts per million and not more than 1.0 parts per million.

### BRIEF DESCRIPTION OF THE DRAWINGS

In the drawings, identical reference numbers identify similar elements or acts. The sizes and relative positions of elements in the drawings may not be necessarily drawn to scale. For example, the shapes of various elements and angles may not be drawn to scale, and some of these elements may be arbitrarily enlarged or positioned to improve drawing legibility. Preferred and alternative embodiments of the present invention are described in detail below with reference to the following drawings:

FIG. 1 is a perspective view of an air purification unit according to an embodiment of the present invention; and

FIG. 2 is a flow diagram of an air purification method according to an embodiment of the present invention.

### DETAILED DESCRIPTION

In the following description, certain specific details are set forth in order to provide a thorough understanding of various embodiments of the invention. However, one skilled in the art will understand that he invention may be practiced without these details. In other instances, well-known structures associated with air purification units, filters, photo-hydro-ionization generators, blowers, fans, controllers, assemblies and subassemblies of the same, and methods of sanitizing one or more indoor spaces by elevating and maintaining a level of ionized-hydro-peroxides, superoxides, hydroxyl ions, or some combination thereof within the indoor space may not necessarily been shown or described in detail to avoid unnecessarily obscuring descriptions of the embodiments of the invention.

In the description, the terms “sanitizing.” “purifying” and “decontaminating,” as well as variants thereof, may be used interchangeably unless stated otherwise, Further, the terms “ambient air,” “indoor space,” “grow space,” and “environment,” as well as variants thereof, may be used interchangeably unless stated otherwise.

The present invention generally relates to methods of using an air purifier to perform a photo-hydro-ionization (PHI) process to generate an oxidizer plasma to be exhausted into an indoor space. The oxidizer plasma may include ionized-hydro-peroxides (IHPs), superoxide, hydroxyl ions or some combination thereof (hereinafter generally referred to as IHPs). In one embodiment, the process sanitizes the ambient environment of the indoor space by purifying air, sanitizing hard surfaces, and decontaminating the environment by creating and maintaining an elevated level of IHPs at or above 0.02 parts per million (ppm) within the entire indoor space.

The processes may be used in environments such as, but not limited to, construction or restoration sites, indoor growing facilities grow houses or green houses), and medical facilities to remove odors, viruses, bacteria, mold spores, mildew, and other bio-contaminants to improve air quality.

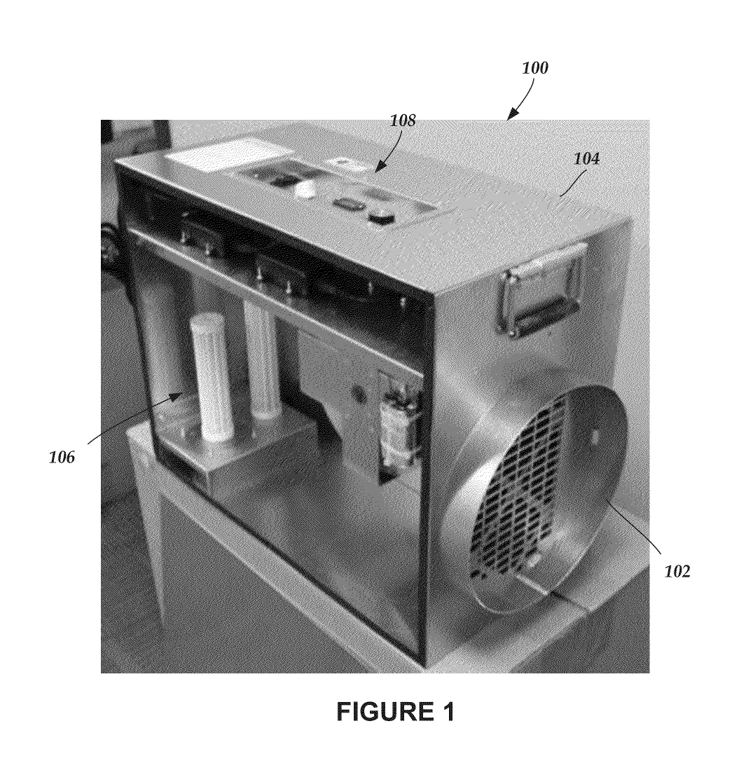

FIG. 1 shows an air purification unit 100 in the form of a stand-alone unit with a variable speed blower 102 to purify air in local or remote volumes or spaces. The unit 100 includes a housing 104 with internal air filters (not shown) on both an intake and an exhaust side of the housing 104 to capture dust, pollen, mold spores. In one embodiment, the unit 100 includes at least one a high-efficiency particulate air (HEPA) filter, which may replace or be in addition to he other filter(s). The housing 104 operates to block out most, if not all, ultraviolet light radiation. The housing 104 includes a motorized impeller (not shown) and at least one PHI generator 106 installed in various orientations such as, but not limited to, vertically and horizontally, but multiple PHI generators may be used to meet purification needs. The unit 100 may include a control panel 108 with a PHI generator selector switch, a timer switch for operation of the PHI generator, a filter replacement light, and a PHI generator gauge or meter. The unit 100 may include a variable speed control for a blower that moves ambient air through the housing 100 at various air flow rates such as, but not limited, to air flow rates in the range of about 100 cubic feet per minute (cfm) to over 2,000 cfm. The unit may also be portable.

The air purification unit 100 may be setup or placed in any indoor area. Air within a first space, whether from an indoor space being sanitized or from a remote location, is drawn in through pre-filters and or hepa filters, then flows over the IHP generator(s) there it mixes with an oxidizer plasma. The oxidizer plasma may include ionized hydroperoxides, superoxides, hydroxyl ions, or some combination thereof. The oxidizer plasma is generated from moisture and oxygen in the surrounding (ambient) air. Purification of contaminated air happens internally within the unit and purified air with additional amounts of free IHP oxidizer plasma is exhausted from the unit where it continues to sanitize the surrounding air and a variety of surfaces within the indoor space. In one embodiment, the unused oxidizer plasma reverts back to Oxygen (0₂) and humidity in about fifteen (15) minutes without leaving a chemical residue behind.

The size of the units, blowers, and the number of PRI generators can be scaled to accommodate different sizes and types of spaces to achieve a desired amount of air purification, which may vary depending on whether the unit is being used in a greenhouse, a grow space or some other space with organic or inorganic matter located therein. The units can be equipped with multiple IHP generators to elevate and maintain the level of IHP inside the entire space to be about 0.02 ppm or greater to instantaneously eliminate odors, volatile organic compounds (VOCs), and bio-contaminates as soon as they are released from plants and soil. The units can be equipped with HEPA filtration combined with HIP generators, to capture mold spores and submicron contaminants. The units may emit only a small amount of green light to minimize or prevent interference with day/night plants cycles.

Growing plants in indoor gardens creates a variety of bio-contaminants, such as mildew, mold spores, viruses, bacteria and VOCs. To minimize or eliminate the bio contaminants and create a healthy environment for the plants, the grow area can he sanitized with HIP. By way of example, an IHP purifier continuously injects ionized hydroperoxide gas into the ambient air and circulates said air around the indoor space (i.e., grow space). The elevated level of IHP provides substantial and nearly instantaneous air purification of the environment.

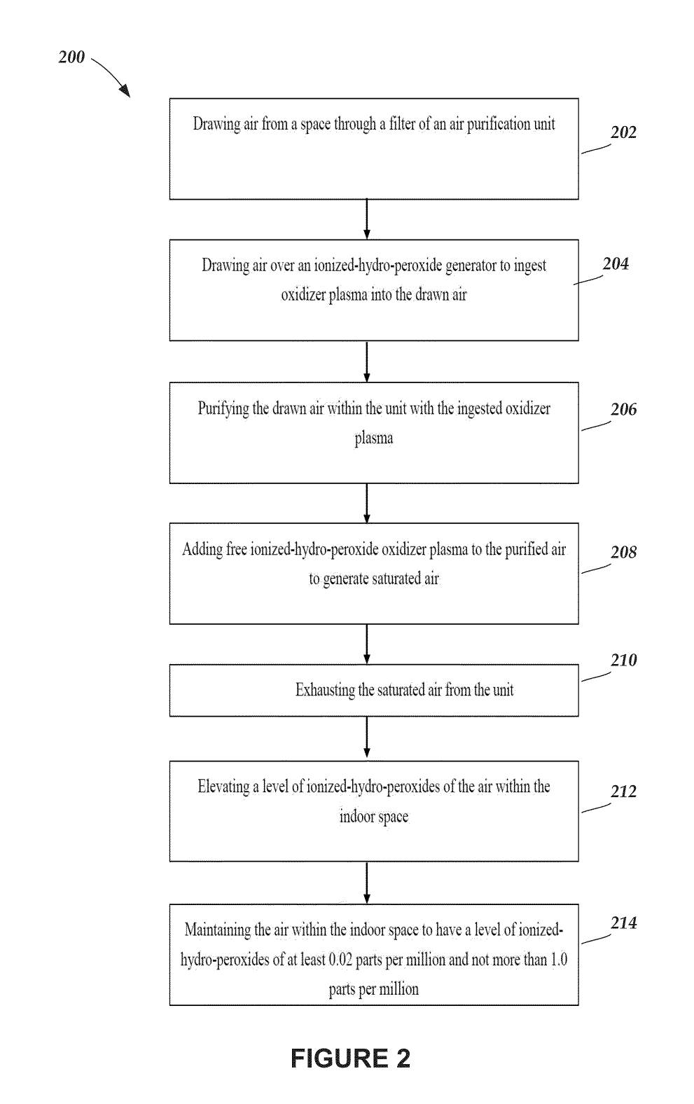

FIG. 2 shows a method 200 for saturating an indoor space with an air purification unit according to an embodiment of the present invention. At Step 202, the unit is activated to draw air from a first space through a filter located within the unit. The filter may take the form of a hepa filter or other types of specialized filters. At Step 204, the air is urged through the unit by a blower and further urged over an ionized-hydro-peroxide (IHP) generator, located in the unit, to impart oxidizer plasma into the drawn air. At Step 206, the IHP generators operate to purify or sanitize the drawn air within the unit vis-à-vis the oxidizer plasma. At Step 208, the HIP generators impart additional free ionized-hydro-peroxide oxidizer plasma into the purified air to generate saturated air (i.e., saturated air includes enough ionized-hydro-peroxide to purify the air within the unit an extra ionized-hydro-peroxide that will move with the purified air that leaves the unit). At Step 210, the saturated air with the extra ionized-hydro-peroxide is exhausted from the unit. At Step 212, the extra ionized-hydro-peroxide is distributed through the indoor space to begin the process of purifying exposed surfaces within the indoor space. At least for some amount of time, depending on a variety of factors (e.g., space size, organic activity, number of surfaces, etc.), an elevated level of ionized-hydro-peroxides will be present in the ambient air of the indoor space. At Step 214 and as the unit continues to operate, the air within the indoor space is maintained to have a level of ionized-hydro-peroxides of at least 0.02 parts per million and not more than 1.0 parts per million.

The various embodiments described above can be combined to provide further embodiments. All of the above U.S. patents, patent applications and publications referred to in this specification are incorporated herein by reference. Aspects can be modified, if necessary, to employ devices, features, and concepts of the various patents, applications and publications to provide yet further embodiments.

These and other changes can be made in light of the above detailed description, in general, in the following claims, the terms used should not be construed to limit the invention to the specific embodiments disclosed in the specification and the claims, but should be construed to include all types of air purifiers, air sanitizers, blowers, IHP generators as well as methods of making, assembling and using the same that operate in accordance with the claims. Accordingly, the invention is not limited by the disclosure, but instead its scope is to be determined entirely by the following claims

1. A method for saturating an indoor space with an air purification unit, the method comprising:
drawing air from a first space through a filter located within the unit;
drawing air over an ionized-hydro-peroxide generator, located in the unit, to impart oxidizer plasma into the drawn air;
purifying the drawn air within the unit with the imparted oxidizer plasma;
adding additional free ionized-hydro-peroxide oxidizer plasma to the purified air to generate saturated air;
exhausting the saturated air from the unit;
elevating a level of ionized-hydro-peroxides of the air within the indoor space; and
maintaining the air within the indoor space to have a level of ionized-hydro-peroxides of at least 0.02 parts per million and not more than 1.0 parts per million.

2. The method of claim 1, further comprising elevating a level of superoxides in the air within the indoor space.

3. The method of claim 1, further comprising elevating a level of hydroxyl ions in the air within the indoor space.

4. The method of claim 1, wherein drawing air from the first space through the filter includes drawing air through a high-efficiency particulate air (HEPA) filter.

5. The method of claim 1, further comprising varying the flow rate of air drawn over the ionized-hydro-peroxide generator using a variable speed blower.

6. The method of claim 1, wherein drawing air from a first space includes drawing air from the indoor space.

7. The method of claim 1, wherein drawing air from a first space includes drawing air from a remote space located outside of the indoor space.

8. The method of claim 1, wherein elevating a level of ionized-hydro-peroxides of the ambient air within the indoor space includes sanitizing at least some of the ambient air and at least a portion of one or more surfaces within the indoor space.
